(19) United States
(12) Patent Application Publication
(10) Pub. No.: US 2010/0331891 A1
(43) Pub. Date: Dec. 30, 2010

(54) SYSTEM AND METHOD FOR SPINAL FIXATION

(75) Inventors: Brad S. Culbert, Rancho Santa Margarita, CA (US); Christopher Warren, Alliso Viejo, CA (US); Robert Flower, Sun City, CA (US); Fausto Olmos, Laguna Niguel, CA (US)

Correspondence Address: KNOBBE MARTENS OLSON & BEAR LLP, 2040 MAIN STREET, FOURTEENTH FLOOR, IRVINE, CA, 92614, US

(73) Assignee: Interventional Spine, Inc., Irvine, CA (US)

(21) Appl. No.: 12/821,980

(22) Filed: Jun. 23, 2010

Related U.S. Application Data
(60) Provisional application No. 61/220,172, filed on Jun. 24, 2009.

(51) Int. Cl. A61B 17/88; A61B 17/86

(52) U.S. Cl. 606/279; 606/301

(57) ABSTRACT

A system and method of bone fixation are provided for improving the bone growth and stability of the fixated bones. For example, a target site for a bone fixation procedure can be accessed at a facet of a first vertebra using a tissue dilator. Bone material can be disrupting from or at the target site, and a bone fixation device can be installed to fix the first vertebra relative to a second vertebra. The disruption and/or removal of the bone material, such as by rasping facets or a facet joint of the first vertebra and the second vertebra, can tend to promote bone growth. Further, it is contemplated that bone graft material can be inserted at the target site, such as into a joint space formed between facets of the first vertebra and the second vertebra.

### CROSS-REFERENCE TO RELATED APPLICATIONS

The present application claims priority under 35 U.S.C. §119(e) to U.S. Provisional Application No. 61/220,172 filed on Jun. 24, 2009, the disclosure of which, including the Appendix, is incorporated by reference herein in its entirety.

### BACKGROUND

1. Field of the Inventions

The present inventions relate to medical devices and, more particularly, to methods and apparatuses for spinal fixation.

2. Description of the Related Art

The human spine is a flexible weight bearing column formed from a plurality of bones called vertebrae. There are thirty-three vertebrae, which can be grouped into one of five regions (cervical, thoracic, lumbar, sacral, and coccygeal). Moving down the spine, there are generally seven cervical vertebrae, twelve thoracic vertebrae, five lumbar vertebrae, five sacral vertebrae, and four coccygeal vertebrae. The vertebrae of the cervical, thoracic, and lumbar regions of the spine are typically separate throughout the life of an individual. In contrast, the vertebra of the sacral and coccygeal regions in an adult are fused to form two bones, the five sacral vertebrae which form the sacrum and the four coccygeal vertebrae which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. The vertebra may be united with various types of fixation systems. These fixation systems may include a variety of longitudinal elements such as rods or plates that span two or more vertebrae and are affixed to the vertebrae by various fixation elements such as wires, staples, and screws (often inserted through the pedicles of the vertebrae). These systems may be affixed to either the posterior or the anterior side of the spine. In other applications, one or more bone screws may be inserted through adjacent vertebrae to provide stabilization.

### SUMMARY

Although spinal fusion is a highly documented and proven form of treatment in many patients, it is contemplated that the rate of bone growth and the quality of the joint formed between fixated bones can be improved. Further, notwithstanding the variety of efforts in the prior art described above, these techniques are associated with a variety of disadvantages. In particular, these techniques typically involve an open surgical procedure, which results in higher cost, lengthy in-patient hospital stays and the pain associated with open procedures. Therefore, there remains a need for improved techniques and systems for stabilization of the spine. Preferably, the devices are implantable through a minimally invasive procedure.

Accordingly, the embodiments of the present inventions provide for apparatuses, methods or performing spinal stabilization, for example, such as posterior lumbar stabilization. In particular, it is contemplated that embodiments disclosed herein can achieve superior fusion of adjacent vertebrae compared to prior art apparatuses and methods. In some embodiments, such improvements are provided through the use of apparatuses and methods that prepare opposing facets of adjacent vertebrae in order to encourage and instigate osseointegration and bone formation at the fusion site.

Various embodiments disclosed herein can be performed during a surgical procedure in which a bone fixation device and/or on graft material is implanted into a given portion of the spinal column. However, it is noted that the methods and apparatuses disclosed herein can also be used in bone fixation procedures other than those occurring in the spinal column. Continuing, various embodiments can comprise decorticating at least a portion of a bone structure used to be fixated relative to their bone structure. Additionally, each of the bone structure is to be fixated can be decorticated. As mentioned, in some embodiments, the decortication of the bone structure can be performed along with the implantation of a bone fixation device and/or bone graft material. It is contemplated that the effectiveness of these fixation procedure and its results can be substantially improved using embodiments disclosed herein.

In accordance with some embodiments, methods of performing bone fixation can comprise accessing a target area, dilating an access path to the target area, and decorticating one or more portions of bone structures to be fixated. Further, such embodiments can also include the steps of implanting a bone fixation device and/or implanting bone graft material. Various apparatuses and methods for implanting bone fixation devices and bone graft material are provided in U.S. Pat. Nos. 5,893,850, 6,511,481, 6,632,224, 6,648,890, 6,685,706, 6,887,243, 6,890,333, 6,908,465, 6,951,561, 7,070,601, and 7,326,211, and U.S. Patent Application Publication Nos. 2004/0260297, 2004/0127906, 2005/0256525, 2006/0030872, 2006/0122609, 2006/0122610, 2007/0016191, 2008/0097436, 2008/0140207, 2008/0306537, the entirety of the disclosures of which are hereby incorporated by reference herein.

In an embodiment, a method of bone fixation is provided that comprises: accessing a target site at a facet of a first vertebra using a tissue dilator; drilling a hole into the facet of the first vertebra; disrupting bone material from or at the target site; and installing a bone fixation device to fix the first vertebra relative to a second vertebra.

In some implementations, the step of disrupting bone material from or at the target site can comprise inserting a rasping device through the tissue dilator to rasp a portion of the first vertebra. Further, the step of disrupting bone material from or at the target site further can comprise rasping a portion of the second vertebra. In some implementations, the step of disrupting bone material from or at the target site can comprise rasping facets of the first vertebra and the second vertebra. For example, the step of disrupting bone material from or at the target site can comprise moving the rasping device in an alternating forward and backward motion within a joint space formed between the facets of the first vertebra and the second vertebra. Furthermore, the step of disrupting bone material from or at the target site further can comprise removing bone material.

In other implementations, the step of installing the bone fixation device can comprise applying secondary compression across the first vertebra and the second vertebra by tensioning the bone fixation device.

Additionally, the method can further comprise the step of inserting bone graft material at the target site. For example, the bone graft material can be inserted into a joint space formed between facets of the first vertebra and the second vertebra.

In accordance with another embodiment, an assembly is provided for performing bone fixation. The assembly can comprise a bone fixation device; an access device for providing an access path to a target site; and a rasping device for disrupting bone material from the target site.

The bone fixation device can comprise a bone screw. The bone fixation device can also comprise a compression screw. The access device can comprise a tissue dilator. In particular, the access device can comprise an expandable access sheath. Additionally, the rasping device can comprise an elongate component having a plurality of teeth. Further, the assembly can further comprise a bone graft funnel instrument for delivering bone graft to the target site.

### BRIEF DESCRIPTION OF THE DRAWINGS

The abovementioned and other features of the inventions disclosed herein are described below with reference to the drawings of the preferred embodiments. The illustrated embodiments are intended to illustrate, but not to limit the inventions. The drawings contain the following figures:

FIGS. 1-3 are perspective, side, and longitudinal cross-sectional views of an embodiment of a fixation device.

FIG. 4 is an enlarged side view of the section 4-4 shown in FIG. 3.

FIG. 5 is a side elevational view of first and second vertebrae and a tissue dilator accessing a target site at the first vertebra, according to an embodiment.

FIG. 6 is an enlarged perspective view of the target site of the first vertebra.

FIG. 7 is an enlarged perspective view of the target site of the first vertebra wherein a distal end of the tissue dilator has been shifted laterally to access a facet joint of the first and second vertebrae, according to an embodiment.

FIG. 8 is an enlarged perspective view of the target site illustrating the rasping of the facet joint, according to an embodiment.

FIG. 9 is a side view of a bone graft funnel instrument, according to an embodiment.

FIGS. 10-11 illustrate elevational views and enlarged views of the target site before and after bone graft material is deployed thereat, according to an embodiment.

FIG. 12 is a rear elevational view of the first and second vertebrae after bone graft material and bone fixation devices have been installed, according to an embodiment.

### DETAILED DESCRIPTION OF THE PREFERRED EMBODIMENT

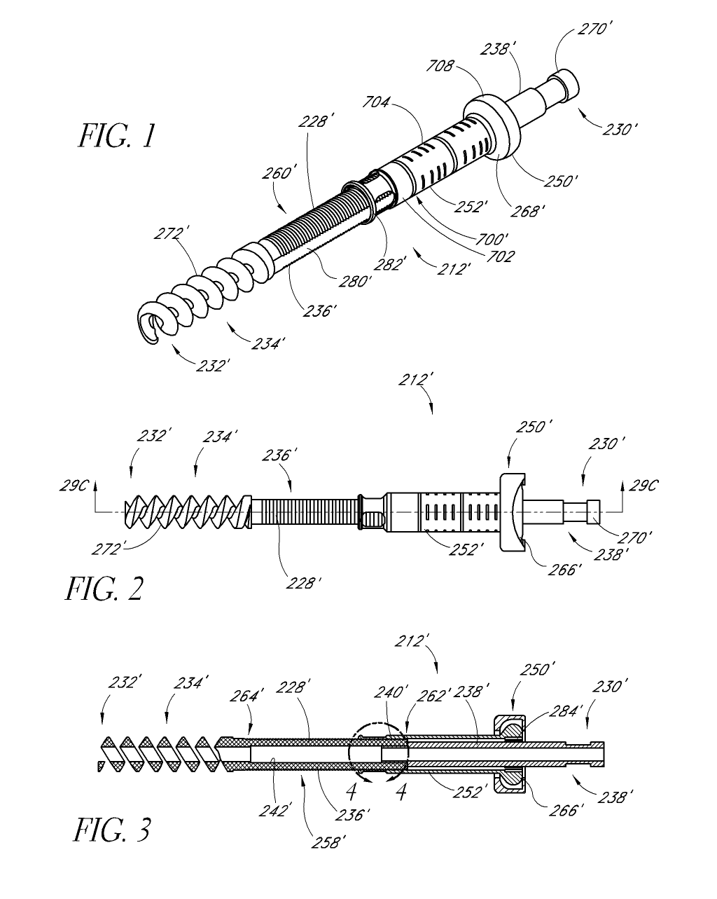

Although the application of the present inventions will be initially disclosed in connection with the spinal fixation devices and procedures illustrated in FIGS. 1-12, the methods and structures disclosed herein are intended for application in any of a wide variety of bones, fixations, and fractures, as will be apparent to those of skill in the art in view of the disclosure herein.

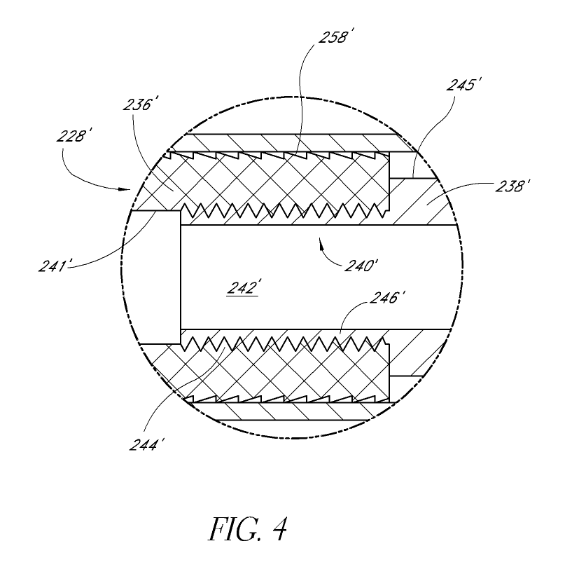

FIGS. 1-4 illustrate an embodiment of a fixation device 212′ having a body 228′ and proximal anchor 700′. In this embodiment, the body 228′ comprises a first portion 236′ and a second portion 238′ that are coupled together at a junction 240′ (FIG. 4). In the illustrated embodiment, the first portion 236′ carries the distal anchor 234′ while the second portion 238′ forms the proximal end 230′ of the body 228′. The first and second portions 236′, 238′ are preferably detachably coupled to each other at the junction 240′. In the illustrated embodiment, the first and second portions 236′, 238′ are detachably coupled to each other via interlocking threads. Specifically, as best seen in FIG. 4, the body 228′ includes an inner surface 241′, which defines a central lumen 242′ that preferably extends from the proximal end 230′ to the distal end 232′ throughout the body 228′. At the proximal end of the first portion 236′, the inner surface 241′ includes a first threaded portion 244′. The first threaded portion 244′ is configured to mate with a second threaded portion 246′, which is located on the outer surface 245′ of the second portion 238′. The interlocking annular threads of the first and second threaded portions 244′, 246′ allow the first and second portions 236′, 238′ to be detachably coupled to each other. In some modified embodiments, the orientation of the first and second threaded portions 244′, 246′ can be reversed. That is, the first threaded portion 244′ can be located on the outer surface of the first portion 236′ and the second threaded portion 246′ can be located on the inner surface 241′ at the distal end of the second portion 238′. Any of a variety of other releasable complementary engagement structures may also be used, to allow removal of second portion 238′ following implantation, as is discussed below.

In a modified arrangement, the second portion 238′ can comprise any of a variety of tensioning elements for permitting proximal tension to be placed on the distal anchor 234′ while the proximal anchor is advanced distally to compress the fracture, fusion site, joint, or bones. For example, any of a variety of tubes or wires can be removably attached to the first portion 236′ and extend proximally to the proximal handpiece. In some such arrangements, the first portion 236′ can include a releasable connector in the form of a latching element, such as an eye or hook. The second portion 238′ can include a complementary releasable connector (e.g., a complementary hook or eye) for engaging the first portion 236′. In this manner, the second portion 238′ can be detachably coupled to the first portion 236′ such that proximal traction can be applied to the first portion 236′ through the second portion as will be explained below. Alternatively, the second portion 238′ may be provided with an eye or hook, or transverse bar, around which or through which a suture or wire may be advanced, both ends of which are retained at the proximal end of the device. Following proximal tension on the tensioning element during the compression step, one end of the suture or wire is released, and the other end may be pulled free of the device. Alternate releasable proximal tensioning structures may be devised by those of skill in the art in view of the disclosure herein.

With particular reference to FIGS. 1-4, the proximal end 230′ of the body 228′ may be provided with a rotational coupling 270′, for allowing the second portion 238′ of the body 228′ to be rotationally coupled to a rotation device. The proximal end 230′ of the body 228′ may be desirably rotated to accomplish one or two discrete functions. In some applications, the proximal end 230′ can be rotated to remove the second portion 238′ of the body 228′ following tensioning of the device across a fracture, fusion site, joint, or bones or to anchor an attachment to the bone. Rotation of the rotational coupling 270′ may also be utilized to rotationally drive the distal anchor into the bone. Any of a variety of rotation devices may be utilized, such as electric drills or hand tools, which allow the clinician to manually rotate the proximal end 230′ of the body. Thus, the rotational coupling 270′ may have any of a variety of cross sectional configurations, such as one or more flats or splines.

With particular reference to FIG. 1, the fixation device may include an antirotation lock between the first portion 236′ of the body 228′ and the proximal anchor 700′. In the illustrated embodiment, the first portion 236′ includes a pair of flat sides 280′, which interact with corresponding flat structures 282′ in the proximal anchor 700′. One or three or more axially extending flats may also be used. As such, rotation of the proximal anchor 700′ is transmitted to the first portion 236′ and the distal anchor 234′ of the body 228′. Of course, those of skill in the art will recognize various other types of splines or other interfit structures can be used to prevent relative rotation of the proximal anchor and the first portion 236′ of the body 228′. For example, in some embodiments, the first portion 236′ may include three flat sides, which interact with corresponding flat structures on the proximal anchor.

To rotate the proximal anchor 700′, the flange 708 is preferably provided with a gripping structure to permit an insertion tool to rotate the flange 708. Any of a variety of gripping structures may be provided, such as one or more slots, flats, bores or the like. In some embodiments, the flange 708 is provided with a polygonal, and, in particular, a pentagonal or hexagonal recess. Further, in FIGS. 2 and 3, the proximal anchor 700′ is shown in combination with a washer 250′. These and other features are shown and described in further detail in U.S. Patent Application Publication Nos. 2004/0127906, 2005/033289, 2007/0118132, and 2007/0123868, the entireties of the disclosures of which are hereby incorporated by reference herein.

Methods of implanting a stabilization device as described above as part of a spinal stabilization procedure will now be described. Although certain aspects and features of the methods and instruments described herein can be utilized in an open surgical procedure, the disclosed methods and instruments are optimized in the context of a percutaneous or minimally invasive approach in which the procedure is done through one or more percutaneous small openings. Thus, the method steps which follow and those disclosed are intended for use in a trans-tissue approach. However, to simplify the illustrations, the soft tissue adjacent the treatment site have not been illustrated in the drawings.

In some embodiments of use, a patient with a spinal instability is identified. Depending upon the spinal fixation technique, the distal ends of one or more bone fixation devices described herein can be advanced into the anterior vertebral body or other suitable portion of one or more vertebrae. As will be explained in more detail below, the fixation device can be used to couple one vertebra that is unstable, separated or displaced to another vertebra, which is not unstable, separated or displaced. However, it should be appreciated that this method may also be applied to three or more vertebrae. In addition, the S-1 portion of the sacrum may be used to stabilize the L5 vertebrae.

The patient is preferably positioned face down on an operating table, placing the spinal column into a normal or flexed position. A trocar optionally may then be inserted through a tissue tract and advanced towards a first vertebrae. In another embodiment, biopsy needle (e.g., Jamshidi™) device can be used. A guidewire may then be advanced through the trocar (or directly through the tissue, for example, in an open surgical procedure) and into the first vertebrae. The guide wire 110 can be inserted into the pedicle of the vertebra preferably through the pars (i.e. the region of the lamina between the superior and inferior articular processes). The trajectory and landmarks of the vertebrae should be considered in performing this step in order to ensure the proper placement of the treatment site, which will provide placement for the guide wire, fixation device, and/or bone graft material.

A suitable expandable access sheath or dilator 112 can then be inserted over the guidewire and expanded to enlarge the tissue tract and provide an access lumen for performing the methods described below in a minimally invasive manner. Using such a device, a surgeon can dilate down to the vertebral level with minimal disruption to the soft tissue while creating a working channel to implant the bone fixation device(s). An example of a device useful for such dilation is the Teleport Tissue Retractor manufactured by Interventional Spine Inc. The Teleport Tissue Retractor is described in co-pending U.S. Patent Application Publication Nos. 2006/0030872 and 2005/0256525, and PCT Publication No. PCT/US2005/027431 (filed as U.S. patent application Ser. No. 11/659,025 on Jan. 30, 2007). In some embodiments, a suitable tissue expander (e.g., a balloon expanded catheter or a series of radially enlarged sheaths) can be inserted over the guidewire and expanded to enlarge the tissue tract. A surgical sheath can then be advanced over the expanded tissue expander. The tissue expander can then be removed such that the surgical sheath provides an enlarged access lumen. Any of a variety of expandable access sheaths or tissue expanders can be used, such as, for example, a balloon expanded catheter, a series of radially enlarged sheaths inserted over each other, and/or the dilation introducer described in U.S. patent application Ser. No. 11/038,784, filed Jan. 19, 2005 (Publication No. 2005/0256525), the entirety of which is hereby incorporated by reference herein.

A drill with a rotatable tip may be advanced over the guidewire and through the sheath. The drill may be used to drill an opening in the first vertebrae. The opening may be configured for (i) insertion of the body 228′ of the bone stabilization device 212′ (such as that shown in FIG. 1), (ii) tapping and/or (iii) providing a counter sink for the proximal anchor 700′. In other embodiments, the step of drilling may be omitted. In such embodiments, the distal anchor 234′ is preferably self-tapping and self-drilling. In embodiments, in which an opening is formed, a wire or other instrument may be inserted into the opening and used to measure the desired length of the body 228′ of the device 212′.

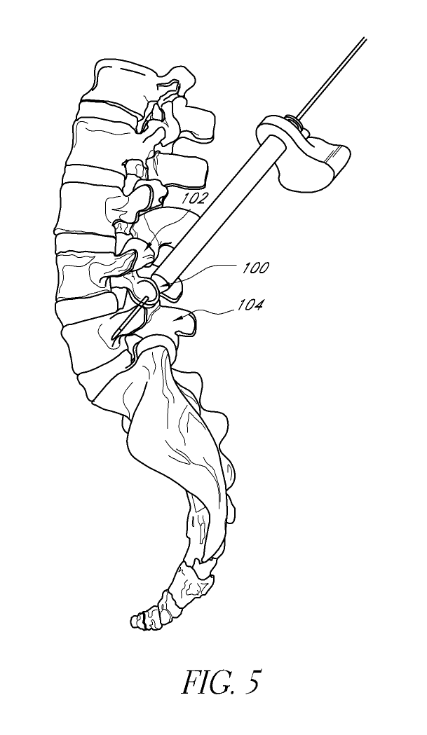

However, in some embodiments, before the bone fixation device is implanted, further preparations to the target site can be made. For example, after the working channel is created in the patient's tissue, the surgeon can then further prepare the target site and the bones that are to be fixated. As discussed herein, embodiments of the methods can be used to fixate adjacent vertebrae and the target site accessed for such procedures can be a facet 100 of the first or superior vertebra 102, as illustrated in FIG. 5. After the working channel has been created to the facet of the first or superior vertebra, the distal end of the tissue dilator can be shifted slightly in order to provide access to the joint where the facet of the first or superior vertebra abuts the facet of the second or inferior vertebra 104.

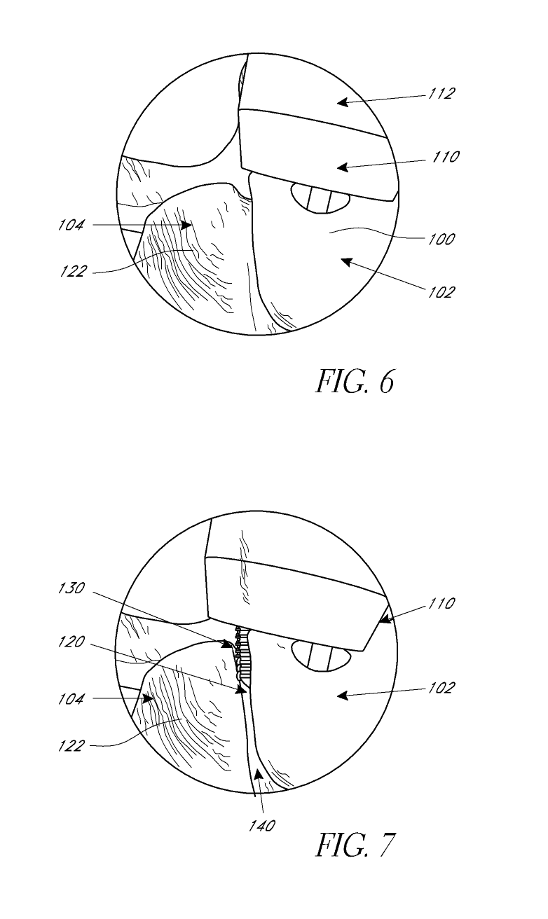

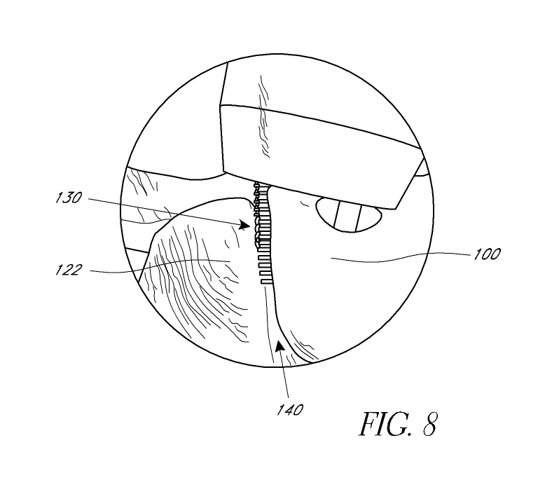

For example, as illustrated in FIG. 6, a distal end 110 of a tissue dilator 112 is initially positioned generally centrally over the facet 100 of the first or superior vertebra 102. In accordance with embodiments disclosed herein, the distal end of the tissue dilator can be shifted slightly laterally from a first position shown in FIG. 6 to a second position shown in FIG. 7 such that the distal end is positioned over a facet joint 120 of the facet 100 of the first or superior vertebra 102 and a facet 122 of the second or inferior vertebra 104.

Once the distal end of the tissue dilator is positioned over the facet joint 120, the facet joint can be accessed by a rasping device 130. The rasping device can be inserted through the tissue dilator until reaching the facet joint. Once at the facet joint, the rasping device can be used to decorticate portions of the facets 100, 122 of the first and second vertebrae. For example, using the tip of the rasping device, the surgeon can cause the rasping device to enter the capsule surrounding the facet joint and advance the rasping device into the joint space 140.

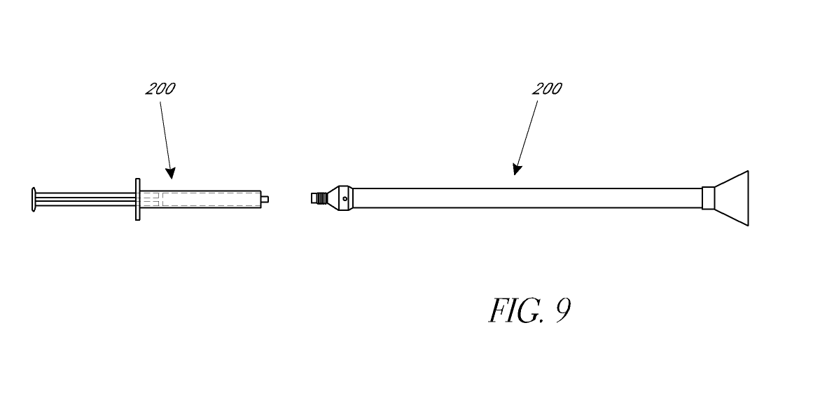

As shown in FIG. 8, once in the joint space, the joint can be decorticated by alternating forward and backward motion on the rasping device in a way that teeth of the rasping device can scrape against the joint faces. If necessary, the rasping device can be repositioned in the plane of the joint to achieve maximum coverage.

The rasping of the joint space serves to decorticate the necessary portions of the adjacent vertebrae in order to promote bone growth and fusion between the vertebrae. Generally, rasping of the joint space will not only disrupt or remove portions of the first and second vertebrae, but will also result in bleeding and exposure of underlying tissue at the joint space. This unique step not only enhances the rate of bone growth, but can also tend to enhance the quality, strength, and effectiveness of the fusion between newly formed bone material and the vertebrae. These distinct advantages represent significant advances over prior art bone fixation methods.

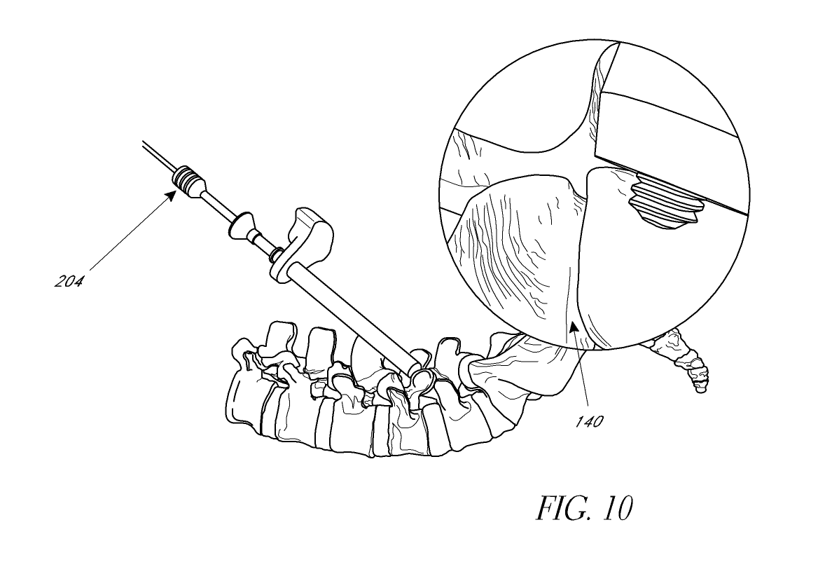

With reference now to FIGS. 9-11, some embodiments can further comprise inserting bone graft material to the target area. For example, the bone graft material can be inserted into the above-noted facet joint in order to promote rapid fixation between the adjacent vertebrae.

Initially, as shown in FIG. 9, bone graft material 200 can be loaded into a funnel instrument 202. Preferably, the graft material can be “backfilled” into the distal end of the funnel as shown. It should also be noted that bone chips and/or autograft must be made into pieces small enough to flow through the funnel. Otherwise, the funnel may become congested and the bone graft may not flow into the joint as desired.

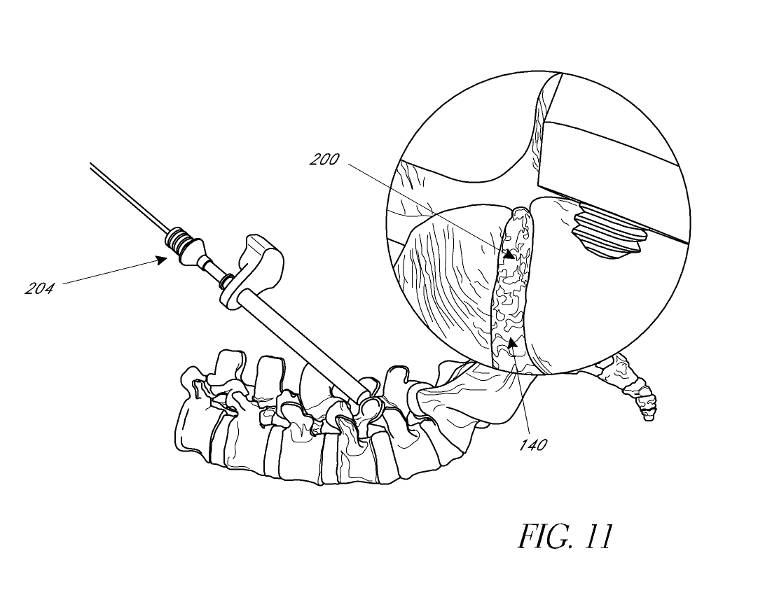

Once the bone graft material is loaded into the final instrument, the bone graft material can be deployed at the target site. As shown in FIG. 10, the funnel instrument can be inserted into the tissue dilator until the distal tip of the funnel instrument is positioned adjacent to the target site. The enlarged inset view of the facet joint and the distal end of the tissue dilator illustrate the target site prior to deployment of bone graft material. The location of the distal tip of the funnel instrument can be modified to any desired location for deploying the graft material at the target site. Then, using a plunger 204 through the funnel instrument, a desired amount of graft material can be injected at the target site. FIG. 11 illustrates the movement of the plunger to deploy the bone graft material into the facet joint at the target site. If using with the PERPOS™ PLS System, manufactured by Interventional Spine Inc., the funnel instrument and plunger can be placed over the k-wire. In such a system, the distal end of the funnel instrument may be rotated clockwise into the hole prepared by the 2-in-1 drill. For example, the funnel may be rotationally seated into or coupled with the pre-drilled (or tapped) bone so that an effective seal is maintained between the funnel and fusion site. The funnel, as shown, includes a thread feature on the distal tip to accomplish this, but a variety of techniques or features may be utilized to achieve. The plunger can then be advanced into the funnel instrument to deploy the graft into the joint.

Further, after the bone fixation device(s) have been inserted, an agent or treatment material (e.g., bone cement) can be delivered from the fixation device 212′ and into the bone proximate to at least a portion of the bone fixation device. The fixation device can be configured to comprise a central lumen and one or more holes or apertures extending outwardly from the central lumen and being in communication with an exterior of the fixation device. In this regard, the agent can be delivered from a proximal end of the device through the lumen, and out through the holes or apertures to the bone proximate the device. The bone cement can set and inhibit, preferably prevent, migration of the fixation device 212′.

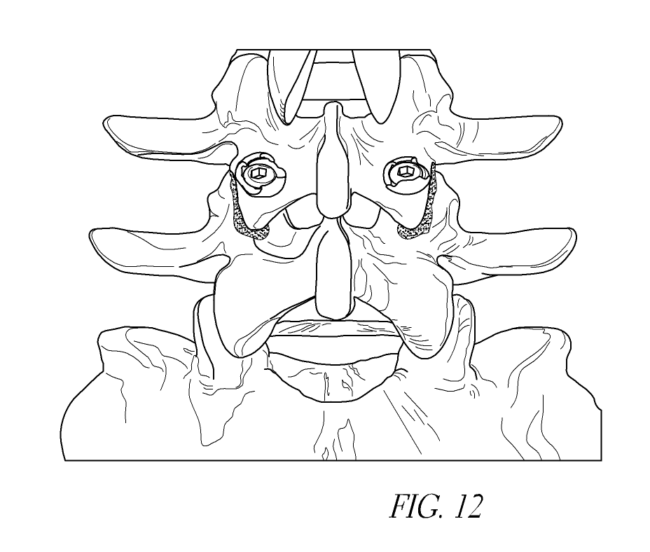

FIG. 12 is a rear perspective view of the adjacent vertebrae after the bone graft material has been inserted at the respective target sites and facet joints and the bone fixation devices have been appropriately tensioned. It is contemplated that in some embodiments, the deployment of the bone graft material at the target site can take place prior to installation of the bone fixation device at the target site. For example, if using the PERPOS™ PLS System, manufactured by Interventional Spine Inc., the bone fixation device can be implanted after the bone graft material has been deployed.

For example, in some embodiments, the body 228′ of the fixation device may be subsequently advanced over the guidewire and through the sheath until it engages the second vertebrae. The body 228′ may be coupled to a suitable insertion tool prior to the step of engaging the fixation device 212′ with the vertebrae. The insertion tool may be configured to engage the coupling 270′ on the proximal end of the body 228′ such that insertion tool may be used to rotate the body 228′. In such an embodiment, the fixation device 212′ is preferably configured such that it can also be advanced over the guidewire.

The insertion tool may be used to rotate the body 228′ thereby driving the distal anchor 234′ to the desired depth within the pedicle of the second vertebrae. The proximal anchor 700 may be carried by the fixation device prior to advancing the body 228′ into the vertebrae, or may be attached and/or coupled to the body 228′ following placement (partially or fully) of the body 228′ within the vertebrae. In another embodiment, the anchor 700 may be pre-attached and/or coupled to the body 228′. In some embodiments, stabilization implants (e.g., a fixation plate and/or rod) may be placed over or coupled to the body or the proximal anchor before the proximal anchor is placed on the body.

In some embodiments, one or more fixation devices may be inserted into the vertebrae with bilateral symmetry such that such two vertebrae are coupled together with two or more fixation devices on a left side of the spine being connected using one or more rods and/or plates to two or more fixation devices on a right side of the spine. In certain of these embodiments, the distal anchor of these fixation devices may be inserted through the pedicle and/or the facet of the vertebrae. In other embodiments, the fixation devices can be utilized to secure adjacent vertebral bodies in combination with another fusion procedure or implant, such as the implantation of a spinal cage, plate or other device for fusing adjacent vertebral bodies. Thus, the fixation devices may operate in conjunction with a cage or other implant to provide three-point stability across a disc space, to assist in resisting mobility between two vertebral bodies. In other embodiments, the fixation device may simply be advanced through a portion of a first vertebra and into a second, preferably adjacent, vertebra. In certain of these embodiments, the fixation device may extend through the facet of the first vertebra and the distal anchor may be inserted through the facet or pedicle of the second vertebra.

In some embodiments, the clinician can have access to an array of devices 212′, having, for example, different diameters, axial lengths, configurations and/or shapes. The clinician can assess the position of the body 228′ with respect to the superior vertebrae and chose the device 212′ from the array, which best fits the patient anatomy to achieve the desired clinical result. In other embodiments, the clinician can have access to an array of devices 212′, having, for example, bodies 228′ of different diameters, axial lengths. The clinician can also have an array of proximal anchors 700, having, for example, different configurations and/or shapes. The clinician can choose the appropriate body 228′ and then assess the position of the body 228′ with respect to the superior vertebrae and choose the proximal anchor 700 from the array, which best fits the patient anatomy to achieve the desired clinical result. In such embodiments, the proximal anchor 700 can be advantageously coupled to body 228′ after the body 228′ is partially or fully inserted into the vertebrae.

Once the distal anchor 234′ is in the desired location, the proximal anchor 700 is preferably advanced over the body 228′ until it reaches its desired position. This may be accomplished by pushing on the proximal anchor 700 or by applying a distal force to the proximal anchor 700. In other embodiments, the proximal anchor 700 can be advanced by applying a proximal retraction force to the proximal end 230′ of body 228′, such as by conventional hemostats, pliers or a calibrated loading device, while distal force is applied to the proximal anchor 700. In this manner, the proximal anchor 700 is advanced distally with respect to the body 228′ until the proximal anchor 700 is in its proper position (e.g., positioned snugly against the outer surface of the vertebra.) Appropriate tensioning of the stabilization device 212′ can be accomplished by tactile feedback or through the use of a calibration device for applying a predetermined load on the stabilization device 212′. As explained above, one advantage of the structure of the illustrated embodiments is the ability to adjust the compression and/or the position of the proximal anchor 700 independently of the setting of the distal anchor 234′ within the vertebra. For example, the positioning of the distal anchor 234′ within the vertebra can be decoupled from the positioning of the proximal anchor 700 with respect to the superior vertebra.

In some embodiments, the proximal anchor 700 can be pushed over the body 228′ by tapping the device with a slap hammer or similar device that can be used over a guidewire. In this manner, the distal end of the device 212′ is advantageously minimally disturbed, which prevents (or minimizes) the threads in the bore from being stripped.

Following appropriate tensioning of the proximal anchor 700, the proximal portion of the body 228′ extending proximally from the proximal anchor 700 can be removed. In some embodiments, this may involve cutting the proximal end of the body 228′. For example, the proximal end of the body may be separated by a cutting instrument or by cauterizing. Cauterizing may fuse the proximal anchor 700 to the body 228′ thereby adding to the retention force between the proximal anchor 700 and the body 228′. Such fusion between the proximal anchor and the body may be particularly advantageous if the pin and the proximal anchor are made from a polymeric or plastic material. In this manner, as the material of the proximal anchor and/or the pin is absorbed or degrades, the fusion caused by the cauterizing can continue to provide retention force between the proximal anchor and the body. In other embodiments, the body can comprise a first and a second portion 236′, 238′ as described above. In such embodiments, the second portion 238′ may be detached from the first portion 236′ and removed. In the illustrated embodiment, this involves rotating the second portion 238′ with respect to the first portion via the coupling 270′. In still other embodiments, the proximal end of the body 228′ may remain attached to the body 228′.

A pair of the fixation devices 12A, 12B may be used to provide stability without additional hardware. In this example, the fixation device 12A, 12B can be used as a trans-facet screw. That is, the fixation device extends through a facet of a first vertebra and into the facet of a second, typically inferior, vertebrae. As in the illustrated embodiment, this procedure is typically (but not necessarily) preformed with bilateral symmetry. Thus, even in the absence of a stabilizing bar tying pedicle screws to adjacent vertebrae or to the sacrum, and in the absence of translaminar screws that can extend through the spinous process, the fixation devices 12A, 12B can be used to stabilize two vertebrae, such as L3 and L4 to each other pending the healing of a fusion. In some embodiments, the body 28 of fixation devices 12A, 12B has a length of approximately 10 mm-30 mm and the diameter of the body is approximately 3 mm to 5.5 mm.

The access site may be closed and dressed in accordance with conventional wound closure techniques and the steps described above may be repeated on the other side of the vertebrae for substantial bilateral symmetry. The bone stabilization devices 12 may be used alone or in combination with other surgical procedures such as laminectomy, discectomy, artificial disc replacement, and/or other applications for relieving pain and/or providing stability.

The specific dimensions of any of the embodiment disclosed herein can be readily varied depending upon the intended application, as will be apparent to those of skill in the art in view of the disclosure herein. Moreover, although the present inventions have been described in terms of certain preferred embodiments, other embodiments of the inventions including variations in the number of parts, dimensions, configuration and materials will be apparent to those of skill in the art in view of the disclosure herein. In addition, all features discussed in connection with any some embodiments herein can be readily adapted for use in other embodiments herein to form various combinations and sub-combinations. The use of different terms or reference numerals for similar features in different embodiments does not imply differences other than those which may be expressly set forth. Accordingly, the present inventions are intended to be described solely by reference to the appended claims, and not limited to the preferred embodiments disclosed herein.

What is claimed is:

1. A method of bone fixation comprising:
accessing a target site at a facet of a first vertebra using a tissue dilator;
drilling a hole into the facet of the first vertebra;
disrupting bone material from or at the target site; and
installing a bone fixation device to fix the first vertebra relative to a second vertebra.

2. The method of claim 1, wherein the step of disrupting bone material from or at the target site comprises inserting a rasping device through the tissue dilator to rasp a portion of the first vertebra.

3. The method of claim 2, wherein the step of disrupting bone material from or at the target site further comprises rasping a portion of the second vertebra.

4. The method of claim 3, wherein the step of disrupting bone material from or at the target site comprises rasping facets of the first vertebra and the second vertebra.

5. The method of claim 3, wherein the step of disrupting bone material from or at the target site comprises moving the rasping device in an alternating forward and backward motion within a joint space formed between the facets of the first vertebra and the second vertebra.

6. The method of claim 1, wherein the step of disrupting bone material from or at the target site further comprises removing bone material.

7. The method of claim 1, wherein the step of installing the bone fixation device comprises applying secondary compression across the first vertebra and the second vertebra by tensioning the bone fixation device.

8. The method of claim 1, further comprising the step of inserting bone graft material at the target site.

9. The method of claim 8, wherein the step of inserting bone graft material at the target site comprises rotationally coupling a funnel instrument with the hole in the facet of the first vertebra.

10. The method of claim 8, wherein the bone graft material is inserted into a joint space formed between facets of the first vertebra and the second vertebra.

11. An assembly for performing bone fixation, the assembly comprising:
a bone fixation device;
an access device for providing an access path to a target site; and
a rasping device for disrupting bone material from the target site.

12. The assembly of claim 11, wherein the bone fixation device comprises a bone screw.

13. The assembly of claim 11, wherein the bone fixation device comprises a compression screw.

14. The assembly of claim 11, wherein the access device comprises a tissue dilator.

15. The assembly of claim 11, wherein the access device comprises an expandable access sheath.

16. The assembly of claim 11, wherein the rasping device comprises an elongate component having a plurality of teeth.

17. The assembly of claim 11, further comprising a bone graft funnel instrument for delivering bone graft to the target site.
